(19) United States
(12) Patent Application Publication
(10) Pub. No.: US 2013/0309202 A1
(43) Pub. Date: Nov. 21, 2013

(54) Methods for Improving Liver Function

(71) Applicant: The Ohio State University, Columbus, OH (US)

(72) Inventors: Chandan K. Sen, New Albany, OH (US); Cameron L. Rink, Galloway, OH (US); Sashwati Roy, New Albany, OH (US); Savita Khanna, New Albany, OH (US)

(73) Assignee: The Ohio State University, Columbus, OH (US)

(21) Appl. No.: 13/897,704

(22) Filed: May 20, 2013

Related U.S. Application Data
(60) Provisional application No. 61/648,782, filed on May 18, 2012.

(51) Int. Cl. A61K 31/353; A61K 45/06

(52) U.S. Cl. CPC A61K 31/353; A61K 45/06
USPC 424/85.7; 514/456; 424/85.4; 514/43

(57) ABSTRACT

The present invention provides methods to improve liver function utilizing tocotrienols. In particular, various liver pathologies may be treated using the present methods, including cirrhosis, hepatitis, and sclerosing cholangtitis. The present invention also provides methods to increase tissue concentrations of tocotrienols.

### CROSS REFERENCE TO RELATED APPLICATIONS

This application claims the benefit of U.S. Provisional Patent Application No. 61/648,782 filed May 18, 2012, the entire disclosure of which is expressly incorporated herein by reference for all purposes.

### STATEMENT REGARDING FEDERALLY SPONSORED RESEARCH

This invention was made with U.S. government support under grant TL1RR025753 and NS42617 from the National Institutes of Health. The government has certain rights in this invention.

### BACKGROUND OF THE INVENTION

The natural vitamin E family is composed of eight members, equally divided into two classes; tocopherols (TCP) and tocotrienols (TE). TCP are characterized by a saturated phytyl side chain with three chiral carbons whereas TE possess a farnesyl side chain with double bonds at carbons 3, 7, and 11. Within each class, isomers are differentiated by α, β, γ, and δ according to the position and degree of methylation on the chromanol head. TCP represent the primary form of vitamin E in green leafy vegetables, while TE are found in highest concentration in seeds of monocotyledons that include the wheat, rice, oat, barley, and palm.

To date, the majority of vitamin E clinical trials have reported negligible or detrimental outcomes across a range of diseases. While these trials address vitamin E as a whole, they have primarily been focused to test only one of eight naturally occurring vitamin E family members, αTCP. With a growing body of literature demonstrating unique biological properties of the lesser-characterized vitamin E family members, the misnomer that αTCP and vitamin E are synonymous represents a blind spot in vitamin E research today. Members of the vitamin E family regulate specific cell signaling pathways independent of their antioxidant properties. αTE suppresses the activity of HMG-CoA reductase, the hepatic enzyme responsible for cholesterol synthesis.

Tocopherol transfer protein (TTP) selectively transports dietary αTCP into tissues. It is commonly held that TTP affinity is a critical determinant for the biological activity of the eight natural vitamin E family members. The affinity of TTP to bind and transport αTE is 12% that of αTCP which has led to the notion that tocotrienol biological activity is negligible. Orally supplemented tocotrienols are transported to vital organs and restore fertility in TTP deficient mice suggesting TTP-independent mechanisms of transport for TE. As the biological importance of TE is increasingly shown, there is a need for evidence-based formulations and methods of administering TE to optimize health and aid in disease management.

Liver disease is a serious condition that can result from many causes and lead to serious complications, including death. Liver conditions may include, for example, hepatitis, cirrhosis, and hepatocellular carcinoma. The Model for End Stage Liver Disease (MELD) scoring system is clinically used to determine the severity of chronic liver disease and to assess the priority and need for liver transplant allocation. The MELD scale ranges from 6 to 40 with the highest scores indicating poor liver function and greater need for a transplantation surgery. The three month mortality of End Stage Liver Disease (ESLD) patients with MELD scores in the range of 10-19, 20-29, 30-39, and over 40 are 6.0%, 19.2%, 52.6%, and 71.3% respectively. There is a need for pharmaceutical or nutraceutical agents to slow ESLD progression; any improvement in the rate of increase in MELD score, or reduction in MELD score, provides more time for a liver transplant candidate to receive a compatible liver.

### SUMMARY OF THE INVENTION

Disclosed herein are methods to improve liver function in a subject with a liver pathology, comprising: a.) administering to a subject with liver pathology at least one tocotrienol selected from the group consisting of: alpha-tocotrienol; beta-tocotrienol; gamma-tocotrienol; and delta-tocotrienol; and b.) improving liver function in the subject as measured by a hepatic function panel test.

Also provided are methods to slow the rise in MELD scores in a subject with liver pathology, comprising: a.) administering to a subject with liver pathology at least one tocotrienol selected from the group consisting of: alpha-tocotrienol; beta-tocotrienol; gamma-tocotrienol; and delta-tocotrienol to a subject with liver pathology; and b.) slowing the rise in MELD scores in the subject.

Also provided are methods for improving prognosis of a subject with liver pathology, comprising: a.) administering to a subject with liver pathology at least one tocotrienol selected from the group consisting of: d-alpha-tocotrienol; d-beta-tocotrienol; d-gamma-tocotrienol; and d-delta-tocotrienol to a subject with liver pathology; and b.) improving prognosis of the subject with liver pathology, as measured by a Model for End-Stage Liver Disease (MELD) score.

Also provided are methods to slow disease progression in a subject with liver pathology, comprising: a.) administering to a subject with liver pathology at least one tocotrienol selected from the group consisting of: alpha-tocotrienol; beta-tocotrienol; gamma-tocotrienol; and delta-tocotrienol; and b.) slowing disease progression in the subject.

Also provided are methods to ameliorate the symptoms of end stage liver disease in a subject with end stage liver disease, comprising: a.) administering to a subject with end stage liver disease at least one tocotrienol selected from the group consisting of: alpha-tocotrienol; beta-tocotrienol; gamma-tocotrienol; and delta-tocotrienol; and b.) ameliorating the symptoms of end stage liver disease in the subject.

Also provided are methods for treating subject with liver disease comprising: administering at least one tocotrienol to a subject with liver disease.

Also provided are methods to increase tissue concentration of at least one tocotrienol in a subject, comprising: a.) administering to a subject at least one tocotrienol selected from the group consisting of: alpha-tocotrienol; beta-tocotrienol; gamma-tocotrienol; and delta-tocotrienol; and b.) increasing tissue concentration of at least one tocotrienol in the subject.

Also provided are methods to increase blood concentration of at least one tocotrienol in a subject, comprising: a.) administering to a subject at least one tocotrienol selected from the group consisting of: alpha-tocotrienol; beta-tocotrienol; gamma-tocotrienol; and delta-tocotrienol; and b.) increasing blood concentration of at least one tocotrienol in the subject.

Also provided are methods to increase skin concentration of at least one tocotrienol in a subject, comprising: a.) administering to a subject at least one tocotrienol selected from the group consisting of: alpha-tocotrienol; beta-tocotrienol; gamma-tocotrienol; and delta-tocotrienol; and b.) increasing skin concentration of at least one tocotrienol in the subject.

Also provided are methods to increase adipose concentration of at least one tocotrienol in a subject, comprising: a.) administering to a subject at least one tocotrienol selected from the group consisting of: alpha-tocotrienol; beta-tocotrienol; gamma-tocotrienol; and delta-tocotrienol; and b.) increasing adipose concentration of at least one tocotrienol in the subject.

Also provided are methods to increase brain concentration of at least one tocotrienol in a subject, comprising: a.) administering to a subject at least one tocotrienol selected from the group consisting of: alpha-tocotrienol; beta-tocotrienol; gamma-tocotrienol; and delta-tocotrienol; and b.) increasing brain concentration of at least one tocotrienol in the subject.

Also provided are methods to increase cardiac muscle concentration of at least one tocotrienol in a subject, comprising: a.) administering to a subject at least one tocotrienol selected from the group consisting of: alpha-tocotrienol; beta-tocotrienol; gamma-tocotrienol; and delta-tocotrienol; and b.) increasing cardiac muscle concentration of at least one tocotrienol in the subject.

Also provided are methods to increase liver concentration of at least one tocotrienol in a subject, comprising: a.) administering to a subject at least one tocotrienol selected from the group consisting of: alpha-tocotrienol; beta-tocotrienol; gamma-tocotrienol; and delta-tocotrienol; and b.) increasing liver concentration of at least one tocotrienol in the subject.

Also provided are methods herein, wherein the subject is intolerant of standard therapeutic measures.

Also provided are methods claim herein, wherein the liver pathology is selected from the group consisting of: cirrhosis; hepatitis; and cholangitis.

Also provided are methods herein, wherein the liver pathology is selected from the group consisting of: viral cirrhosis; alcoholic cirrhosis; infectious cirrhosis; autoimmune cirrhosis; decompensated cirrhosis;

Also provided are methods herein, wherein the tocotrienol is administered according to Table A.

Also provided are methods herein, wherein the tocotrienol is administered according to Table B.

Also provided are methods herein, wherein the tocotrienol is administered according to Table C.

Also provided are methods herein, wherein the tocotrienol is administered according to Table D.

Also provided are methods herein, wherein the tocotrienol is administered according to Table E.

Also provided are methods herein, which further comprises administering an additional pharmaceutical composition.

Also provided are methods herein, which further comprises administering a composition selected from the group consisting of: peginterferon; alfa-2b; and ribavirin.

Also provided are methods herein, which further comprises measuring the tocotrienol concentration in a tissue from the subject, wherein the tissue is selected from the group consisting of: blood; skin; adipose; brain; cardiac muscle; and liver.

Also provided are methods herein, wherein tissue concentration of at least one tocotrienol is increased by a multiplier selected from the group consisting of about: 1.2×; 1.3×; 1.4×; 1.5×; 1.6×; 1.7×; 1.8×; 1.9×; 2×; 3×; 4×; 5×; 6×; 7×; 8×; 9×; 10×; 11×; 12×; 13×; 14×; and 15×.

Also provided are methods herein, wherein the tocotrienol administered comprises tocopherol, by weight percent of total, less than a percent selected from the group consisting of: 50%; 40%; 30%; 20%; 15%; 10%; 5%; and 1%.

Also provided are methods herein, wherein the tocotrienol is substantially free of tocopherol.

Also provided are methods herein, wherein tissue concentration of at least one tocotrienol after administration is selected from the group consisting of about: at least about 0.5 μm/L to at least about 50 μm/L; at least about 1 μm/L to at least about 40 μm/L; at least about 2 μm/L to at least about 30 μm/L; at least about 3 μm/L to at least about 25 μm/L; at least about 4 μm/L to at least about 20 μm/L; and at least about 5 μm/L to at least about 15 μm/L.

Also provided are methods herein, wherein adipose tissue concentration of at least one tocotrienol after administration is selected from the group consisting of about: at least about 4 μm/L to at least about 25 μm/L; at least about 5 μm/L to at least about 25 μm/L; at least about 6 μm/L to at least about 20 μm/L; at least about 7 μm/L to at least about 15 μm/L; at least about 8 μm/L to at least about 15 μm/L; and at least about 9 μm/L to at least about 15 μm/L.

Also provided are methods herein, wherein brain tissue concentration of at least one tocotrienol after administration is selected from the group consisting of about: at least about 0.2 μm/L to at least about 1.9 μm/L; at least about 0.25 μm/L to at least about 1.8 μm/L; at least about 0.3 μm/L to at least about 1.7 μm/L; at least about 0.4 μm/L to at least about 1.6 μm/L; at least about 0.5 μm/L to at least about 1.5 μm/L; and at least about 0.6 μm/L to at least about 1.5 μm/L.

Also provided are methods herein, wherein heart tissue concentration of at least one tocotrienol after administration is selected from the group consisting of about: at least about 0.3 μm/L to at least about 15 μm/L; at least about 0.3 μm/L to at least about 14 μm/L; at least about 0.4 μm/L to at least about 12 μm/L; at least about 0.5 μm/L to at least about 10 μm/L; at least about 0.7 μm/L to at least about 9 μm/L; and at least about 0.8 μm/L to at least about 7 μm/L.

Also provided are methods herein, wherein liver tissue concentration of at least one tocotrienol after administration is selected from the group consisting of about: at least about 0.01 μm/L to at least about 5 μm/L; at least about 0.25 μm/L to at least about 2 μm/L; at least about 0.03 μm/L to at least about 1 μm/L; at least about 0.1 μm/L to at least about 0.8 μm/L; at least about 0.2 μm/L to at least about 0.7 μm/L; and at least about 0.3 μm/L to at least about 0.6 μm/L.

Also provided are methods herein, wherein the tocotrienol is derived from at least one plant selected from the group consisting of: wheat; rice; barley; and palm.

Also provided are methods herein, wherein the tocotrienol is derived from palm oil.

Also provided are methods herein, wherein the tocotrienol is Tocovid SupraBio.

Also provided are methods to treat end stage liver disease in a patient with end stage liver disease, comprising: a.) administering at least one daily dose of tocotrienol formulation to a patient with end stage liver disease, wherein the tocotrienol formulation comprises approximately 123 mg d-alpha tocotrienol; approximately 16 mg d-beta tocotrienol; approximately 225 mg d-gamma tocotrienol; and approximately 51 mg d-delta tocotrienol; and b.) treating end stage liver disease in the patient.

Also provided are methods to treat cirrhosis in a patient with cirrhosis, comprising: a.) administering at least one daily dose of tocotrienol formulation to a patient with cirrhosis, wherein the tocotrienol formulation comprises approximately 123 mg d-alpha tocotrienol; approximately 16 mg d-beta tocotrienol; approximately 225 mg d-gamma tocotrienol; and approximately 51 mg d-delta tocotrienol; and b.) treating cirrhosis in the patient.

Also provided are methods to treat viral hepatitis in a patient with viral hepatitis, comprising: a.) administering at least one daily dose of tocotrienol formulation to a patient with viral hepatitis, wherein the tocotrienol formulation comprises approximately 123 mg d-alpha tocotrienol; approximately 16 mg d-beta tocotrienol; approximately 225 mg d-gamma tocotrienol; and approximately 51 mg d-delta tocotrienol; and b.) treating viral hepatitis in the patient.

Also provided are methods to treat primary sclerosing cholangitis in a patient with primary sclerosing cholangitis comprising: a.) administering at least one daily dose of tocotrienol formulation to a patient with primary sclerosing cholangitis, wherein the tocotrienol formulation comprises approximately 123 mg d-alpha tocotrienol; approximately 16 mg d-beta tocotrienol; approximately 225 mg d-gamma tocotrienol; and approximately 51 mg d-delta tocotrienol; and b.) treating primary sclerosing cholangitis in the patient.

Also provided are methods to hepatitis C in a patient with hepatitis C, comprising: a.) administering at least one daily dose of tocotrienol formulation to a patient with hepatitis C, wherein the tocotrienol formulation comprises approximately 123 mg d-alpha tocotrienol; approximately 16 mg d-beta tocotrienol; approximately 225 mg d-gamma tocotrienol; and approximately 51 mg d-delta tocotrienol; and b.) treating hepatitis C in the patient.

Also provided are methods to treat hepatitis B in a patient with hepatitis B, comprising: a.) administering at least one daily dose of tocotrienol formulation to a patient with hepatitis B, wherein the tocotrienol formulation comprises approximately 123 mg d-alpha tocotrienol; approximately 16 mg d-beta tocotrienol; approximately 225 mg d-gamma tocotrienol; and approximately 51 mg d-delta tocotrienol; and b.) treating hepatitis B in the patient.

### BRIEF DESCRIPTION OF THE FIGURES

FIG. 1A-1B. Human whole blood αTE (FIG. 1A) and αTCP (FIG. 1B) concentration following oral TE supplementation. Data represent individual values (males n=6, females n=10) and mean±SD at baseline (0 wk), 6 wk and 12 wk. Within each treatment group, levels without a common letter differ, P<0.05.

FIG. 2A-2B. Human skin αTE (FIG. 2A) and αTCP (FIG. 2B) concentration following oral TE supplementation. Data represent individual values (males n=6, females n=10) and mean±SD at baseline (0 wk) and 12 wk. Within each treatment group, levels without a common letter differ, P<0.05.

FIG. 3A-3C. Fitted MELD scores of TCP patients (FIG. 3A), TE patients (FIG. 3B), and mean fitted MELD scores of TCP and TE supplemented patients (FIG. 3C) relative to length of vitamin E supplementation. Data represent the progression of MELD score relative to time. No significant difference was found in the mean fitted MELD score slope prior to supplementation. Within each post-supplementation group, mean fitted MELD score slopes without a common letter differ, P<0.05.

FIG. 4A-4C. Human blood α-TE, γ-TE, and α-TCP concentration following oral TE supplementation.

FIG. 5A-5C. Human skin TE and TCP concentration following oral TE supplementation.

### DETAILED DESCRIPTION OF THE INVENTION

The present invention is based, in part, on the discovery that oral TE supplementation increased αTE in every vital organ tested, including the liver. Oral TE supplementation increases tissue levels beyond therapeutic levels showing that dietary TE intake and supplementation play an important role in human health.

As in other vital organs tested, the initial goal of collecting liver from transplant patients was to determine tissue TE content following long-term oral supplementation. On the basis of clinical feedback that TE supplemented patients had a slower rise in the model for end stage liver disease (MELD) scores compared to TCP supplemented patients, the inventors studied the significance of TE on MELD score outcomes in end stage liver disease (ESLD) patients.

The MELD score was introduced in 1999 to quantify the prognosis of cirrhotic patients after trans-jugular intrahepatic portosystemic shunt. The MELD scale ranges from 6 to 40 with the highest scores indicating poor liver function and greater need for a transplantation surgery. MELD uses the patient's values for serum bilirubin, serum creatinine, and the international normalized ratio for prothrombin time (INR) to predict survival. The three month mortality of ESLD patients with MELD scores in the range of 10-19, 20-29, 30-39, and over 40 are 6.0%, 19.2%, 52.6%, and 71.3% respectively.

End Stage Liver Disease. In the current study, 50% of ESLD participants receiving oral TE supplementation had a reduction in their MELD score. In contrast, a study by Huo et. al. demonstrated that participants receiving standard of care treatment only had a 16% reduction in MELD over time.

Standard of care data. A review of recent literature on ESLD reveals the potential clinical impact of the present invention. Of 124 ESLD patients evaluated in a study to assess variability of MELD score during the year before transplantation, only one patient of the 124 reviewed had a reduction in MELD score greater than 5. MELD score is a reliable marker for mortality

Viral Hepatic Cirrhosis. The effect of oral TE in attenuating the time-dependent rise of MELD was most evident in patients with viral hepatic cirrhosis.

Viral Hepatitis. Oral TE supplementation demonstrated an effect in patients with viral hepatitis. Of ESLD patients in the TE supplemented group, 4 out of 6 participants with hepatitis C and the sole subject with hepatitis B had a reduced MELD score following treatment.

Standard of care data: The significance of the present invention is highlighted by a study comparing MELD scores in hepatitis C cirrhosis patients treated with or without standard of care therapy. Of 129 patients eligible, 66 received peginterferon, alfa-2b, and ribavirin for 24 wk while 63 patients received no treatment. MELD scores for treated patients decreased significantly after 24 wk of therapy (14.1+/−2.9 vs. 10.5+/−2.3) while patients in the untreated control group had an increase in MELD score (14.5+/−3.4 vs. 16.7+/−3.2). However, only 27 patients in the treated group tolerated therapy, 26 patients had their dose reduced due to toxicity, and 13 patients had treatment discontinued due to intolerance. Despite such adverse effects, in decompensated cirrhotics, hepatitis C virus clearance by therapy is life-saving and reduces disease progression.

Adjuvant Therapy. TE in adjunctive therapy to either slow disease progression or to allow a reduction in therapy in patients who do not tolerate standard therapeutic measures is within the scope of the present invention.

Tissue and Organ Availability of TE. The present invention discloses the tissue availability of TE in vital organs of adult humans following oral supplementation, and characterizes multiple vital organ concentration of TCP in adults. Patients supplemented for even the shortest duration had detectable levels of TE in tissue. That TE was delivered and accumulated in vital human organs demonstrates that oral TE supplementation enriches its concentration in whole blood, adipose, skin, brain, cardiac muscle, and liver.

TABLE A
Tocotrienol daily dose, by milligrams:

| Tocotrienol | Range 1 | Range 2 | Range 3 | Range 4 |
|---|---|---|---|---|
| alpha | 100-150 | 110-140 | 115-130 | 120-125 |
| gamma | 180-270 | 190-260 | 200-250 | 220-230 |
| delta | 35-70 | 40-65 | 45-60 | 48-54 |

TABLE B
Tocotrienol w/w percent, by total tocotrienols:

| Tocotrienol | Range 1 | Range 2 | Range 3 | Range 4 |
|---|---|---|---|---|
| alpha | 0-50 | 20-40 | 25-35 | 28-32 |
| gamma | 0-70 | 45-65 | 50-60 | 54-58 |
| delta | 0-25 | 5-20 | 8-15 | 10-14 |

TABLE C
Tocotrienol, by doses per day:

| Tocotrienol | Range 1 | Range 2 | Range 3 | Range 4 |
|---|---|---|---|---|
| alpha | 0-6 | 1-5 | 2-4 | 1-2 |
| gamma | 0-6 | 1-5 | 2-4 | 1-2 |
| delta | 0-6 | 1-5 | 2-4 | 1-2 |

TABLE D
Tocotrienol doses, by number of days per week:

| Tocotrienol | Range 1 | Range 2 | Range 3 | Range 4 |
|---|---|---|---|---|
| alpha | 0-7 | 1-6 | 2-5 | 6-7 |
| gamma | 0-7 | 1-6 | 2-5 | 6-7 |
| delta | 0-7 | 1-6 | 2-5 | 6-7 |

TABLE E
Tocotrienol doses, by number of weeks per year:

| Tocotrienol | Range 1 | Range 2 | Range 3 | Range 4 |
|---|---|---|---|---|
| alpha | .5-52 | 1-20 | 4-20 | 4-12 |
| gamma | .5-52 | 1-20 | 4-20 | 4-12 |
| delta | .5-52 | 1-20 | 4-20 | 4-12 |

### EXAMPLES

### Example 1

### Experimental Methods

Human Participants

The study protocol was reviewed and approved by the institutional review board of The Ohio State University. All patients provided written informed consent. Due to limitations in obtaining healthy adult human tissue, whole blood and skin biopsy samples were taken from the Healthy Participants Group while vital organ tissue was acquired from the Surgical Patients Group.

Healthy Participants Group

Whole blood and skin vitamin E concentration were compared at baseline (pre-supplementation) to samples collected after 12 wk of supplementation with TE. Healthy participants (n=16) received 400 mg of TE daily. Adult volunteers provided two skin biopsy and three blood samples. Skin biopsies were collected from the right (1^{st} biopsy at 0 wk) and left (2^{nd} biopsy at 12 wk) inner thigh. Whole blood was taken at 0, 6 and 12 wk. Healthy participants were chosen for this study because they could be supplemented for a defined time period (not bound by scheduled surgery as in the Surgical Patients Group). This allowed the inventors to collect pre-supplementation baseline samples. In this group participants were not supplemented with TCP as each participant was naive to TE and acted as their own control. Inclusion criteria for the Healthy Participants Group included: age 21-40 years old, good health, non-smoker, non-pregnant or non-breastfeeding, and no recent (past 6 mo) or current use of supplements containing vitamin E. Exclusion criteria for the Healthy Participants Group included: diabetes or HIV infection, receiving immunosuppression therapy, neurological disease, and alcohol or drug use.

Surgical Patients Group

Adult surgical patients were randomized to supplementation of either 400 mg TCP or 400 mg TE daily. Vital organs for study included: cardiac muscle acquired from heart transplant recipients with end stage heart failure (TCP n=3, TE n=5); liver from transplant recipients with end-stage liver (TCP n=3, TE n=4); adipose acquired from abdominal adipose tissue of morbidly obese patients undergoing reconstructive plastic surgery (TCP n=4, TE n=5); and brain tissue from recalcitrant epilepsy patients requiring resection (TE n=4). Control brain samples were taken from autopsy participants donated to science and represent vitamin E concentrations of the general population, without dietary TE consumption (n=4). Exclusion critera inculded current or recent dietary supplementation of vitamin E and surgical patients under 21 y of age. Both TCP and TE supplemented groups received comparable physician prescribed diets which did not include additional dietary supplements.

Supplementation Regimen and Compliance

For the current study, vitamin E capsules were supplied by Carotech Inc., 21 Balmoral Court, Talmadge Village, Edison, N.J. 08817, USA. The entire study was conducted using vitamin E gel capsules manufactured in a single batch and immediately shipped to the inventors. Capsule content was validated using a sensitive coulometric electrode detection method developed by the inventors' laboratory.

The Surgical Patients Group participants were randomized to receive either 400 mg TE (200 mg Tocovid SupraBio b. i. d.) or 400 mg TCP (200 mg b.i.d.). The Healthy Participants Group received only 400 mg TE (200 mg b.i.d.). A single 200 mg Tocovid SupraBio softgel capsule contains 61.52 mg d-alpha-tocotrienol, 8.11 mg d-beta-tocotrienol; 112.8 mg d-gamma-tocotrienol, and 25.68 mg d-delta-tocotrienol. TCP gel capsules contained 200 mg of d-alpha-tocopherol. Vitamin E gel capsules were sealed in blister packs. To determine compliance, study participants mailed empty packages back to the clinic every two weeks. Participant supplementation compliance for the study was >90%.

Supplementation length for surgical groups was determined by the initiation of vitamin E supplementation to the day before scheduled surgery. For all surgical patients, a minimum of 4 wk of supplementation was desired. However, in some cases physician-directed necessity of surgery did not permit a full 4 wk. Tissue specific mean, minimum, and maximum length of supplementation for patients is reported in Supplemental Table 1.

SUPPLEMENTAL TABLE 1
Length of vitamin E supplementation¹

| Organ | n² | days |
|---|---|---|
| Blood | | |
| TCP | 16 | 84 ± 0 (84-84) |
| TE | 16 | 84 ± 0 (84-84) |
| Skin | | |
| TCP | 16 | 84 ± 0 (84-84) |
| TE | 16 | 84 ± 0 (84-84) |
| Adipose | | |
| TCP | 4 | 83 ± 55 (14-145) |
| TE | 5 | 157 ± 75 (88-280) |
| Brain | | |
| TCP | 4 | NA³ |
| TE | 4 | 261 ± 278 (78-672) |
| Heart | | |
| TCP | 3 | 14 ± 5 (9-18) |
| TE | 5 | 155 ± 167 (30-443) |
| Liver | | |
| TCP | 3 | 144 ± 16 (129-161) |
| TE | 4 | 181 ± 129 (8-309) |

¹Values are mean ± SD (range)
²For adipose, brain, heart, and liver n represents patients that went to surgery, not total enrollment
³Autopsy tissue used - no supplementation

Vitamin E Extraction and Analyses

Excised tissues were minced, rinsed in phosphate buffered saline to remove blood, and stored in liquid nitrogen until analysis. Vitamin E extraction was performed using a highly sensitive HPLC-coulometric electrode array detector (CoulArray Detector Model 5600 with 12 channels; ESA Inc., Chelmsford, Mass., USA).

### Example 2

### Statistical Analysis

Healthy Participants Group

Box plots were used to determine outliers; defined as values greater than the 75 percentile plus 1.5 times the inter-quartile range or values less than the 25 percentile minus 1.5 times the inter-quartile range. Twelve outliers were identified and it was determined that lab procedural errors were the cause and thus removed from the analysis. Random-effects linear regression was used to compare the concentrations for vitamin E isoforms across weeks of TE supplementation for both the blood and the skin samples. If the overall P-value was significant at the 0.05 level, the inventors subsequently compared 0 vs. 6 wk, 0 vs. 12 wk, and 6 vs. 12 wk. The P-values were adjusted using the Holm's procedure to conserve the overall type I error at 5%. For skin samples, the inventors compared 0 vs. 12 wk of supplementation with TE. Gender was included as an effect modifier (interaction with weeks of supplementation). If the interaction covariate was not significant, then gender was included as a main effect. Again, if gender by itself was not significant it was removed from the regression model. The vitamin E isoforms were transformed using the natural logarithm in order to normalize the values within groups and to stabilize the variance across groups. This is a typical assumption when using random-effects linear regression. Data represent individual values for men, women, as well as the mean±SD for men, women, and both sexes taken together. P<0.05 was considered significant.

Surgical Patients Group

Summary statistics for vitamin E concentration in adipose, brain, cardiac muscle, and liver of surgical patients are presented according to supplementation group (TE or TCP). Wilcoxon rank-sum was used to test differences across vitamin E supplementation for the 5 detectable vitamin E isoforms. Non-parametric analysis (Wilcoxon rank-sum) was used due to small sample sizes; between 2 and 5 observations. P<0.05 was considered significant. The US RDA is based on nutrient level that is sufficient for 97-98% of the population; therefore data are presented as percentile values.

Model of End-Stage Liver Disease Score Analysis

Random-effects linear regression was used to estimate the individual slope and intercepts of the MELD score pre- and post-supplementation for each subject. This was performed separately for TCP and TE supplementation groups. Random-effects regression takes into account the variability within participants due to repeated measures and the variability between participants in order to estimate the standard error. Due to the serendipitous nature of the MELD score findings the length of supplementation was not standardized between patients awaiting liver transplantation. The time scale is in days relative to beginning of the patient's vitamin E supplementation. The estimated slopes presented in the results were multiplied by 10,000 since the MELD score change is relatively small compared to the change in days of observations (1,000 to 1,500 d). The percent change in the slope from pre- to post-supplementation was calculated. Summary statistics are presented for pre- and post-slope and the percent change across TE and TCP supplementation groups. Wilcoxon rank-sum was used to test differences in slope and percent change in slope between TCP and TE. Wilcoxon signed-rank test was used to compare pre- to post-supplementation. P value<0.05 was considered significant. All statistical analyses were run using Stata 10.1 software (Stata Corporation, College Station, Tex.).

### Example 3

### Experimental Results

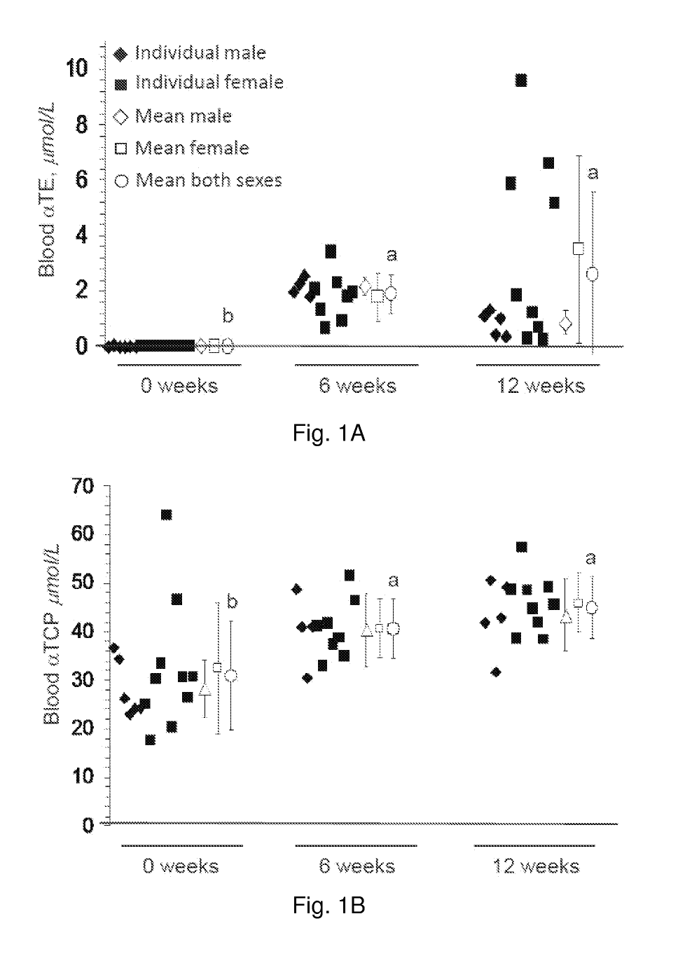

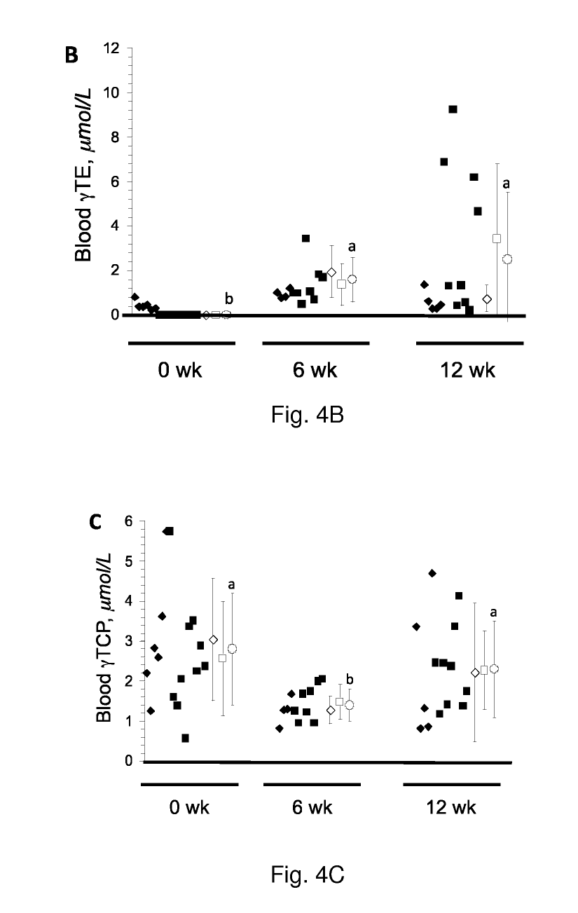

In peripheral whole blood of non-supplemented humans, baseline TE levels were negligible. TE supplementation significantly increased the concentration of TE in peripheral blood of both men and women (FIG. 1A and FIGS. 4A, 4B). The mean concentration of αTE in whole blood of TE supplemented participants was more than 1.5 μmol/L following 6 wk and 2.5 μmol/L following 12 wk of supplementation (FIG. 1A). TE supplementation also significantly increased whole blood αTCP levels in study participants. TE supplementation modestly decreased whole blood γTCP⁹ levels following 6 wk of supplementation. However, after 12 wk, the concentration did not differ from baseline. The data presented demonstrates that daily oral supplementation of TE in a typical human diet is significantly effective in increasing the concentration of tocotrienols in peripheral blood.

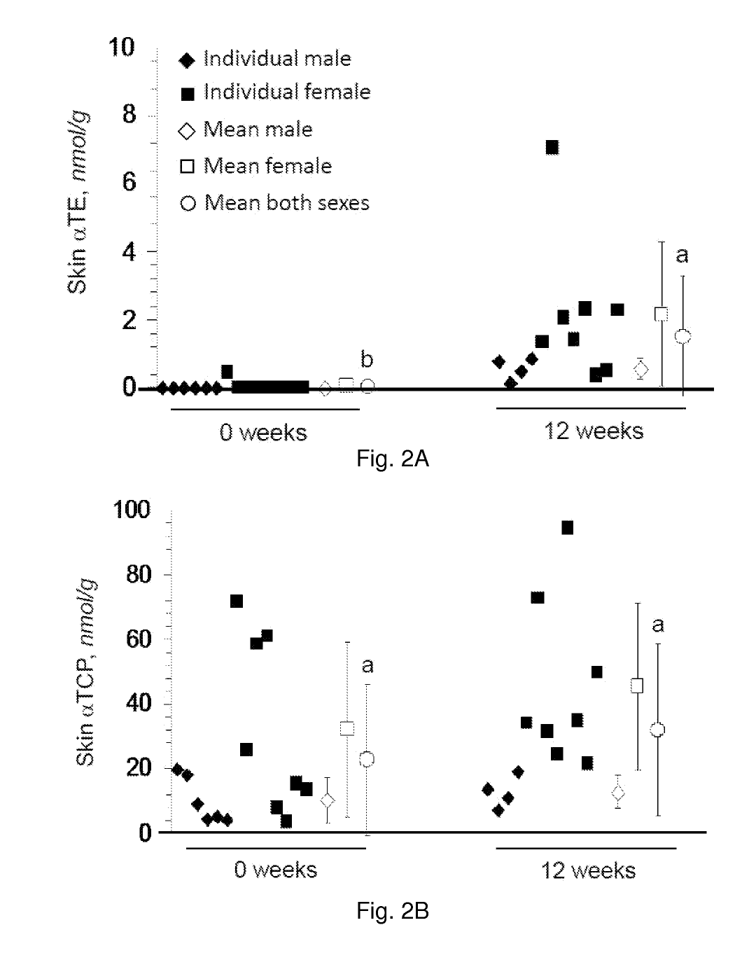

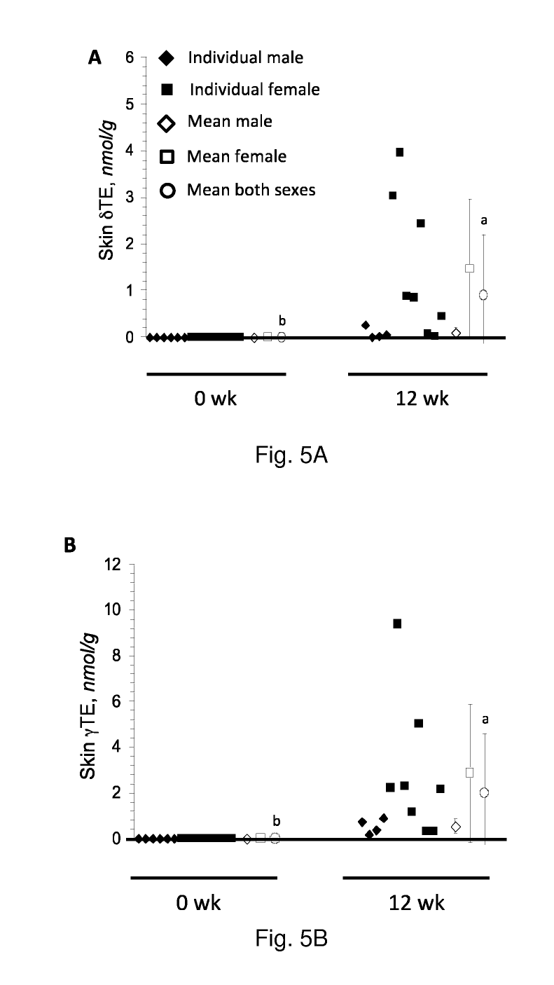

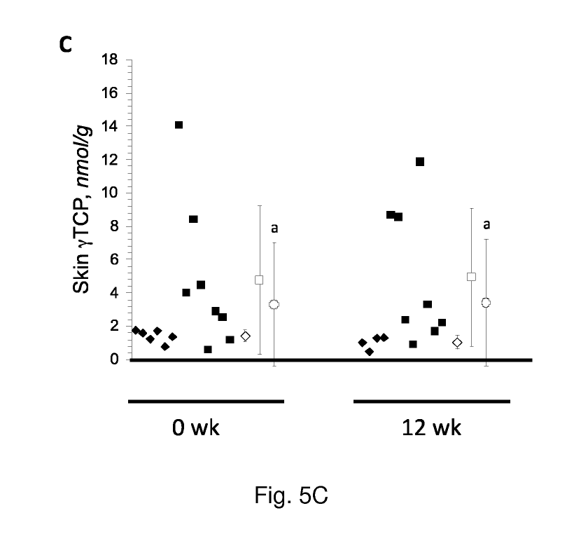

As in whole blood, only trace baseline amounts of αTE, γTE, and δTE were detected in the skin of healthy participants not supplemented with TE (FIGS. 2A, 2B and FIGS. 5A, 5B). Following 12 wk of TE supplementation, skin concentration of αTE, γTE, and δTE was significantly elevated. Combined data for males and females showed a significant increase in all three isoforms at 12 wk. Oral TE supplementation had no significant effect on αTCP or γTCP skin concentration.

Adipose tissue emerged as reservoir for TE in supplemented humans. The abdominal adipose concentration of TE supplemented patients was significantly greater than in the other vital organs studied (Table 1). The adipose αTE, γTE and δTE concentrations were ˜10-fold greater than in controls (P<0.05). The ratio of αTE to αTCP in adipose of TE supplemented participants was 1:4, as compared to 1:25 in patients receiving TCP alone. TE supplementation had no discernible effect on adipose tissue tocopherol concentration (Table 1).

TABLE 1
Adipose vitamin E concentration¹

| | percentile | | | | |
|---|---|---|---|---|---|
| | 25^{th} | 50^{th} | 75^{th} | mean | P value² |
| | nmol/g | | | | |
| TCP supplemented | | | | | |
| αTCP | 13.2 | 23.6 | 44.1 | 28.6 | |
| γTCP | 5.57 | 13.2 | 21.0 | 13.3 | |
| αTE | 0.30 | 0.66 | 1.61 | 0.95 | |
| γTE | 0.71 | 1.72 | 3.21 | 1.96 | |
| δTE | 0.05 | 0.50 | 1.36 | 0.71 | |
| TE supplemented | | | | | |
| αTCP | 24.5 | 25.9 | 35.5 | 36.7 | 0.462 |
| γTCP | 4.47 | 8.29 | 9.36 | 8.84 | 0.462 |
| αTE | 7.49 | 7.89 | 13.6 | 9.94 | 0.028* |
| γTE | 12.4 | 17.1 | 23.5 | 17.2 | 0.014* |
| δTE | 6.82 | 7.00 | 11.9 | 8.73 | 0.028* |

¹TCP supplemented n = 4, TE supplemented n = 5. Sample size is smaller than total number of patients enrolled as not all patients went to surgery.
²P value from Wilcox rank-sum test comparing each isoform across supplementation group,
*P < 0.05.

Trace levels of TE were detected in control brain tissue. TE supplementation significantly elevated αTE, γTE, and δTE concentrations in the human brain (Table 2). Participants supplemented with TE had a significantly lower level of αTCP then cadaveric brains (Table 2). In heart muscle, αTE, γTE, and δTE levels were significantly higher in TE supplemented patients as compared to participants receiving TCP alone (Table 3). No statistical difference was observed in heart α- and γTCP levels between treatment groups (Table 3). TE supplementation also increased liver αTE, γTE, and δTE concentration significantly as compared to patients supplemented with TCP (Table 4). However, similar to prior small animal research that examined dietary TE supplementation, hepatic αTE concentration was markedly lower than αTCP in the liver of TE supplemented patients Unlike heart muscle and adipose tissues, TCP supplemented patients had significantly higher αTCP concentrations in liver tissue as compared to their TE counterparts (Table 4). While the concentration of αTE, γTE, and δTE in liver tissue was less than 10% that found in adipose (Table 4), each isoform was detected in liver of TE supplemented participants.

TABLE 2
Brain vitamin E concentration¹

| | percentile | | | | |
|---|---|---|---|---|---|
| | 25^{th} | 50^{th} | 75^{th} | mean | P value² |
| | nmol/g | | | | |
| TCP supplemented | | | | | |
| αTCP | 41.1 | 43.3 | 60.0 | 50.5 | |
| γTCP | 0.98 | 2.51 | 10.6 | 5.77 | |
| αTE | 0.03 | 0.04 | 0.05 | 0.04 | |
| γTE | 0.03 | 0.05 | 0.08 | 0.06 | |
| δTE | 0.09 | 0.15 | 0.17 | 0.13 | |
| TE supplemented | | | | | |
| αTCP | 24.3 | 33.0 | 38.5 | 31.4 | 0.043* |
| γTCP | 1.25 | 2.87 | 3.77 | 2.51 | 0.564 |
| αTE | 0.71 | 0.80 | 1.87 | 1.29 | 0.021* |
| γTE | 0.47 | 0.70 | 1.19 | 0.83 | 0.021* |
| δTE | 0.24 | 0.38 | 0.67 | 0.45 | 0.021* |

¹TCP supplemented n = 4, TE supplemented n = 4. Sample size is smaller than total number of patients enrolled as not all patients went to surgery.
²P value from Wilcox rank-sum test comparing each isoform across supplementation group,
*P < 0.05.

TABLE 3
Heart vitamin E concentration¹

| | percentile | | | | |
|---|---|---|---|---|---|
| | 25^{th} | 50^{th} | 75^{th} | mean | P value² |
| | nmol/g | | | | |
| TCP supplemented | | | | | |
| αTCP | 15.0 | 26.9 | 53.9 | 31.9 | |
| γTCP | 2.23 | 10.3 | 13.7 | 8.76 | |
| αTE | 0.08 | 0.08 | 0.12 | 0.09 | |
| γTE | 0.02 | 0.23 | 0.26 | 0.17 | |
| δTE | 0.05 | 0.06 | 0.17 | 0.09 | |
| TE supplemented | | | | | |
| αTCP | 30.7 | 33.9 | 45.3 | 45.0 | 0.999 |
| γTCP | 5.06 | 5.35 | 17.6 | 13.1 | 0.655 |
| αTE | 1.70 | 6.23 | 7.25 | 5.37 | 0.025* |
| γTE | 2.07 | 6.70 | 14.4 | 8.85 | 0.025* |
| δTE | 0.70 | 1.70 | 3.39 | 2.32 | 0.053 |

¹TCP supplemented n = 3, TE supplemented n = 5. Sample size is smaller than total number of patients enrolled as not all patients went to surgery.
²P value from Wilcox rank-sum test comparing each isoform across supplementation group,
*P < 0.05.

TABLE 4
Liver vitamin E concentration¹

| | percentile | | | | |
|---|---|---|---|---|---|
| | 25^{th} | 50^{th} | 75^{th} | mean | P value² |
| | nmol/g | | | | |
| TCP supplemented | | | | | |
| αTCP | 48.0 | 68.0 | 77.3 | 64.3 | |
| γTCP | 4.71 | 4.75 | 8.67 | 6.04 | |
| αTE | ND* | ND | ND | ND | |
| γTE | ND | ND | ND | ND | |
| δTE | ND | ND | ND | ND | |
| TE supplemented | | | | | |
| αTCP | 17.7 | 29.5 | 34.3 | 26.0 | 0.033* |
| γTCP | 2.24 | 3.90 | 4.9 | 3.6 | 0.157 |
| αTE | 0.05 | 0.37 | 0.78 | 0.42 | 0.028* |
| γTE | 0.17 | 0.45 | 1.1 | 0.61 | 0.028* |
| δTE | 0.04 | 0.16 | 0.38 | 0.21 | 0.079 |

¹TCP supplemented n = 3, TE supplemented n = 4. Sample size is smaller than total number of patients enrolled as not all patients went to surgery.
²P value from Wilcox rank-sum test comparing each isoform across supplementation group, *P < 0.05.
³ND = not detected, a numerical value of 0 was assigned to ND.

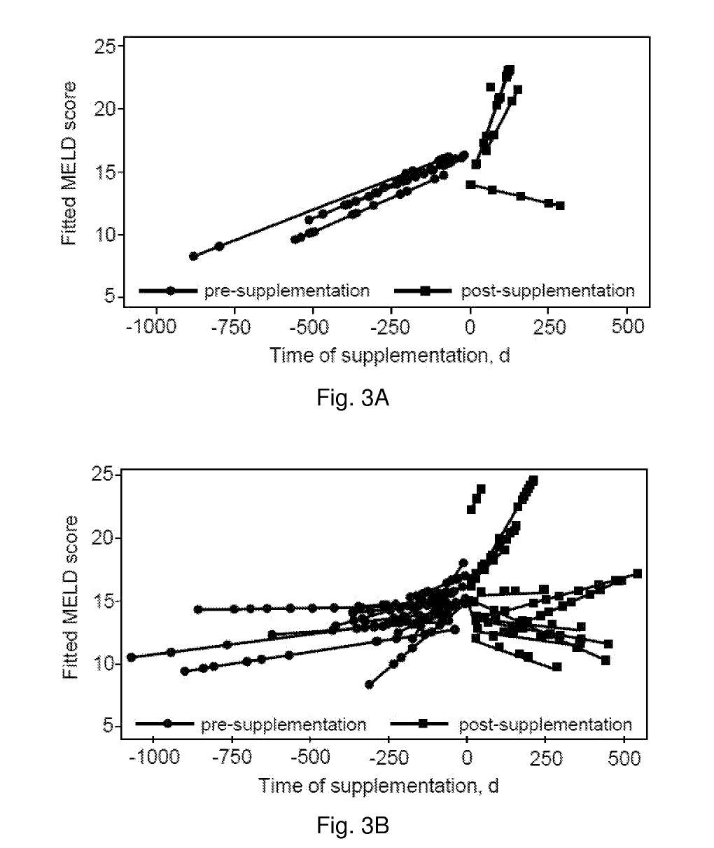

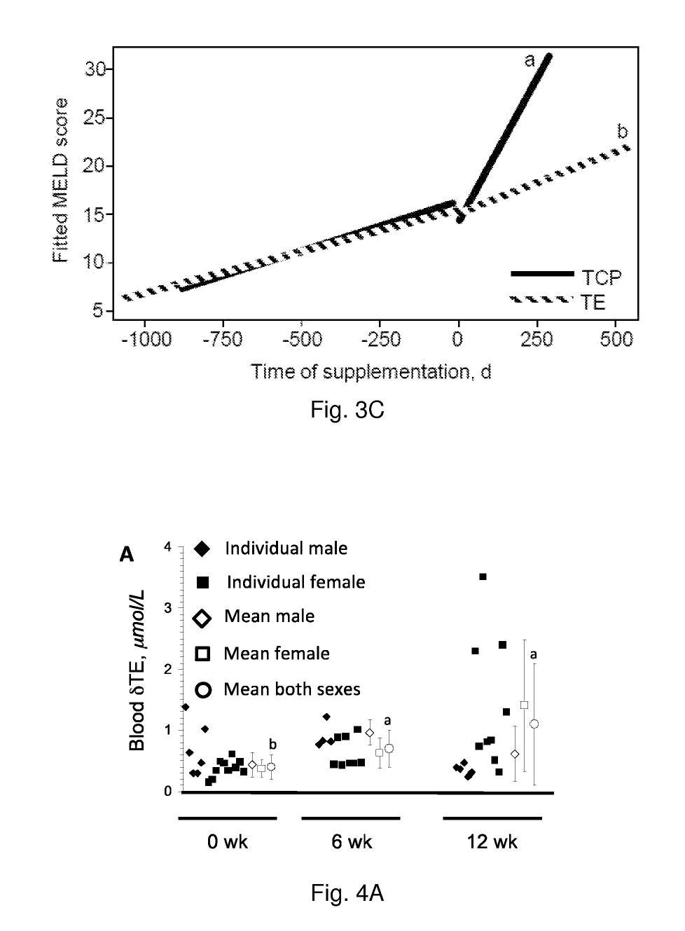

The MELD scoring system is clinically used to determine the severity of chronic liver disease and to assess the priority and need for liver transplant allocation. Oral TE supplementation blunted the time-dependent rise in MELD score as compared to TCP supplemented patients. Of participants supplemented with TCP, only one patient (20%) showed improvement (i.e. lowering) of MELD score (FIG. 3A). In contrast, seven of the fourteen (50%) participants supplemented with TE had a reduction in MELD score (FIG. 3B). Indeed, the slope of the mean fitted MELD score over time for TE supplemented patients was significantly less than that of TCP supplemented patients (FIG. 3C). This effect was most evident in patients with viral hepatic cirrhosis. When stratified on the basis of liver disease diagnosis, TE supplementation lowered the MELD score in 4 of 6 (67%) hepatitis C patients, and the single hepatitis B patient (Supplemental Table 2).

SUPPLEMENTAL TABLE 2
Meld Score Slope¹

| Treatment | Pre-supplement | | Post-supplement | | | |
|---|---|---|---|---|---|---|
| group | Intercept | Slope | Intercept | Slope | % Change | Diagnosis |
| TCP | 16.5 | 101 | 14.4 | 1139 | 1028 | Cirrhosis secondary to postnecrotic |
| | | | | | | autoimmune lupoid band |
| TCP | 16.9 | 98 | 14.2 | 702 | 619 | Primary sclerosing cholangitis |
| TCP | 16.4 | 102 | 14.2 | 712 | 598 | Cirrhosis secondary to NASH |
| TCP | 16.4 | 102 | 14.2 | 483 | 372 | Hepatitis C cirrhosis |
| TCP | 15.7 | 109 | 14.0 | −60 | −155 | Portal hypertension |
| TE | 15.4 | 15 | 15.8 | 430 | 2729 | Hepatitis C cirrhosis |
| TE | 16.2 | 70 | 16.4 | 244 | 252 | Cryptogenic cirrhosis |
| TE | 17.4 | 106 | 16.6 | 282 | 167 | Cryptogenic cirrhosis |
| TE | 14.9 | 4 | 15.7 | 10 | 163 | Hepatitis C cirrhosis |
| TE | 14.6 | 29 | 13.6 | 70 | 137 | Cryptogenic cirrhosis |
| TE | 16.6 | 123 | 16.7 | 274 | 122 | Primary sclerosing cholangitis |
| TE | 18.7 | 372 | 21.7 | 542 | 46 | Cirrhosis - no further information |
| TE | 15.5 | 126 | 11.6 | 108 | −14 | Hepatitis B cirrhosis |
| TE | 15.2 | 212 | 15.0 | 120 | −43 | Primary sclerosing cholangitis |
| TE | 14.3 | 32 | 13.9 | −26 | −182 | Hepatitis C cirrhosis |
| TE | 15.3 | 26 | 13.9 | −45 | −271 | Laennec's cirrhosis (alcoholic) |
| TE | 13.8 | 19 | 12.9 | −35 | −280 | Hepatitis C cirrhosis |
| TE | 13.1 | 39 | 12.3 | −89 | −330 | Hepatitis C cirrhosis |
| TE | 16.2 | 34 | 15.5 | −111 | −422 | Hepatitis C cirrhosis |

¹Patients awaiting liver transplantation were supplemented with 400 mg TCP or TE daily as described in methods. Data represent pre- and post-supplementation MELD intercept, slope, percent change of slope (pre vs. post) and ESLD diagnosis.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims. Whenever a range is given in the specification, all intermediate ranges and sub-ranges, as well as all individual values included in the ranges given are intended to be included in the disclosure. When a Markush group or other grouping is used herein, all individual members of the group and all combinations and sub-combinations possible of the group are intended to be individually included in the disclosure.

In general the terms and phrases used herein have their art-recognized meaning, which can be found by reference to standard texts, journal references and contexts known to those skilled in the art. The above definitions are provided to clarify their specific use in the context of the invention.

What is claimed is:

1. A method to improve liver function in a subject with a liver pathology, comprising:
a.) administering to a subject with liver pathology at least one tocotrienol selected from the group consisting of: d-alpha-tocotrienol; d-beta-tocotrienol; d-gamma-tocotrienol; and d-delta-tocotrienol; and
b.) improving liver function in the subject, as measured by hepatic function panel test.

2. A method for improving prognosis of a subject with liver pathology, comprising:
a.) administering to a subject with liver pathology at least one tocotrienol selected from the group consisting of: d-alpha-tocotrienol; d-beta-tocotrienol; d-gamma-tocotrienol; and d-delta-tocotrienol to a subject with liver pathology; and
b.) improving prognosis of the subject with liver pathology, as measured by a Model for End-Stage Liver Disease (MELD) score.

3. A method to slow disease progression in a subject with liver pathology, comprising:
a.) administering to a subject with liver pathology at least one tocotrienol selected from the group consisting of: d-alpha-tocotrienol; d-beta-tocotrienol; d-gamma-tocotrienol; and d-delta-tocotrienol; and
b.) slowing disease progression in the subject.

4. A method for treating a subject with liver disease comprising: administering at least one tocotrienol to a subject with liver disease.

5. The method of claim 4 wherein the subject has end stage liver disease, and wherein the treating comprises:
a.) administering to a subject with end stage liver disease at least one tocotrienol selected from the group consisting of: d-alpha-tocotrienol; d-beta-tocotrienol; d-gamma-tocotrienol;
and d-delta-tocotrienol; and
b.) ameliorating the symptoms of end stage liver disease.

6. The method of claim 4, further comprising:
a.) administering to a subject at least one tocotrienol selected from the group consisting of: d-alpha-tocotrienol; d-beta-tocotrienol; d-gamma-tocotrienol; and d-delta-tocotrienol; and
b.) increasing liver tissue concentration of at least one tocotrienol in the subject.

7. The method of claim 4, wherein the subject is intolerant of standard therapeutic measures.

8. The method of claim 2, wherein the MELD score in the subject is compared to a control and the subject's MELD score is lower than the control.

9. The method of claim 8, wherein the control is a composite of MELD scores of patients with liver disease at a stage corresponding to the disease stage of the subject.

10. The method of claim 8, wherein the subject's MELD score increases at a rate lower than the control.

11. The method of claim 2 wherein the subject's MELD score stabilizes within 24 weeks of the initiation of a TE therapy.

12. The method of claim 4, wherein the subject's MELD score decreases within 24 weeks of the initiation of a TE therapy.

13. The method of claim 4, wherein the liver disease is a pathology is selected from the group consisting of: cirrhosis; hepatitis; and cholangitis.

14. The method of claim 4, wherein the liver pathology is selected from the group consisting of: hepatocellular carcinoma; viral cirrhosis; alcoholic cirrhosis; infectious cirrhosis; autoimmune cirrhosis; decompensated cirrhosis; cryptogenic cirrhosis; viral hepatitis; hepatitis C; hepatitis B; and primary sclerosing cholangitis.

15. The method of claim 4, wherein the tocotrienol is administered according to a dose of at least one of: Table A; Table B; Table C; Table D; and Table E.

16. The method of claim 4, which further comprises administering an additional pharmaceutical composition.

17. The method of claim 4, which further comprises administering a composition selected from the group consisting of: peginterferon; alfa-2b; and ribavirin.

18. The method of claim 1, further comprising: measuring the tocotrienol concentration in a tissue from the subject, wherein the tissue is selected from the group consisting of: blood; skin; adipose; brain; cardiac muscle; and liver.

19. The method of claim 1, wherein tissue concentration of at least one tocotrienol is increased by a multiplier selected from the group consisting of about: 1.2×; 1.3×; 1.4×; 1.5×; 1.6×; 1.7×; 1.8×; 1.9×; 2×; 3×; 4×; 5×; 6×; 7×; 8×; 9×; 10×; 11×; 12×; 13×; 14×; and 15×.

20. The method of claim 4, wherein the tocotrienol administered comprises tocopherol, by weight percent of total, less than a percent selected from the group consisting of: 50%; 40%; 30%; 20%; 15%; 10%; 5%; and 1%.

21. The method of claim 4, wherein the tocotrienol is substantially free of tocopherol.

22. The method of claim 1, wherein tissue concentration of at least one tocotrienol after administration is selected from the group consisting of about: at least about 0.5 μm/L to at least about 50 μm/L; at least about 1 μm/L to at least about 40 μm/L; at least about 2 μm/L to at least about 30 μm/L; at least about 3 μm/L to at least about 25 μm/L; at least about 4 μm/L to at least about 20 μm/L; and at least about 5 μm/L to at least about 15 μm/L.

23. The method of claim 1, wherein adipose tissue concentration of at least one tocotrienol after administration is selected from the group consisting of about: at least about 4 μm/L to at least about 25 μm/L; at least about 5 μm/L to at least about 25 μm/L; at least about 6 μm/L to at least about 20 μm/L; at least about 7 μm/L to at least about 15 μm/L; at least about 8 μm/L to at least about 15 μm/L; and at least about 9 μm/L to at least about 15 μm/L.

24. The method of claim 1, wherein brain tissue concentration of at least one tocotrienol after administration is selected from the group consisting of about: at least about 0.2 μm/L to at least about 1.9 μm/L; at least about 0.25 μm/L to at least about 1.8 μm/L; at least about 0.3 μm/L to at least about 1.7 μm/L; at least about 0.4 μm/L to at least about 1.6 μm/L; at least about 0.5 μm/L to at least about 1.5 μm/L; and at least about 0.6 μm/L to at least about 1.5 μm/L.

25. The method of claim 1, wherein heart tissue concentration of at least one tocotrienol after administration is selected from the group consisting of about: at least about 0.3 μm/L to at least about 15 μm/L; at least about 0.3 μm/L to at least about 14 μm/L; at least about 0.4 μm/L to at least about 12 μm/L; at least about 0.5 μm/L to at least about 10 μm/L; at least about 0.7 μm/L to at least about 9 μm/L; and at least about 0.8 μm/L to at least about 7 μm/L.

26. The method of claim 1, wherein liver tissue concentration of at least one tocotrienol after administration is selected from the group consisting of about: at least about 0.01p m/L to at least about 5 μm/L; at least about 0.25 μm/L to at least about 2 μm/L; at least about 0.03 μm/L to at least about 1 μm/L; at least about 0.1 μm/L to at least about 0.8 μm/L; at least about 0.2 μm/L to at least about 0.7 μm/L; and at least about 0.3 μm/L to at least about 0.6 μm/L.

27. The method of claim 1, wherein the tocotrienol is derived from at least one plant selected from the group consisting of: wheat, rice, oat, barley, and palm.

28. The method of claim 1, wherein the tocotrienol is derived from palm oil.

29. The method of claim 1, wherein the tocotrienol is Tocovid SupraBio.

30. The method of claim 4, further comprising:
a.) administering at least one daily dose of tocotrienol formulation to a patient in need thereof, wherein the tocotrienol formulation comprises approximately 123 mg d-alpha tocotrienol; approximately 16 mg d-beta tocotrienol; approximately 225 mg d-gamma tocotrienol; and approximately 51 mg d-delta tocotrienol; and
b.) treating liver disease in the patient, wherein the liver disease is selected from the group consisting of: end stage liver disease; cirrhosis; viral hepatitis; and primary sclerosing cholangitis.
